(19) United States
(12) Patent Application Publication
(10) Pub. No.: US 2003/0124025 A1
(43) Pub. Date: Jul. 3, 2003

(54) Biologically safe mailbox

(76) Inventors: Gary W. Mize, Leesville, LA (US); Brandy O. Mize, Leesville, LA (US)

Correspondence Address: Michael S. Neustel, Suite No. 4, 2534 South University Drive, Fargo, ND, 58103, US

(21) Appl. No.: 10/039,804

(22) Filed: Jan. 3, 2002

(51) Int. Cl. A61L 9/00; B01J 19/00; G01D 11/26; B01D 11/04; A61L 2/00

(52) U.S. Cl. 422/28; 422/1; 422/3; 422/31; 422/32; 422/40; 422/119; 422/256; 422/292; 422/300

(57) ABSTRACT

A method and apparatus is provided to protect the mail carrier and mail recipient from exposure to mail contaminated with biological agents. A bio-safe device is user programmed and loaded with an inexpensive decontaminant to automatically perform a treatment cycle on deposited mail to eliminate anthrax and other infectious agents from within an enclosed mail receptacle. This provides the delivery mail carrier or an individual mail recipient the means to destroy infectious agents before physical contact with the mail.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to the decontamination of biological agents. More specifically, the present invention provides a method and apparatus to destroy biological agents such as Anthrax carried in the mail.

2. Description of the Prior Art

Biological warfare, also known as bioterrorism, is the intentional use of organisms to harm or kill people. Terrorists are most likely to use organisms that cause infectious diseases because they are easily spread among people. Disease causing organisms include Yersinia pestis (bubonic plague), tularemia (a plague like bacterial infection), clostridium botulinum (botulism) and tuberculosis but are unlikely to cause widespread disease because they are difficult to manufacture and distribute. Smallpox is more likely choice for bioterrorism since it can spread very rapidly from person to person. Smallpox is passed when infected people sneeze, spraying fine droplets of the virus into the air or through direct contact such as close contact or kissing. Experts on biological warfare regard the bacterium Bacillus anthraces (anthrax) as the biggest hazard.

Microbiologists consider anthrax a serious hazard because it has characteristics that make it suitable as a weapon. It is produced easily and is readily available around the world. The spores do not require special handling procedures so terrorists could take anthrax to many points for distribution. Unlike other infectious agents such as smallpox, the anthrax spores can survive severe heat and cold. Anthrax can infect people through superficial cuts or wounds, the intestine after the consumption of infected food and the lungs after inhalation of spores.

An early diagnosis of anthrax is difficult where the symptoms are similar to those seen with flu; fever, chills and muscle aches. Anthrax resulting from inhalation of spores is the form of illness that would likely occur with a bioterrorist attack and would initially resemble a viral respiratory illness and then would progress to severe shortness of breath and hypoxia, a low concentration of oxygen in the blood.

Following the attack and destruction of the World Trade Center in New York, anthrax has been detected in the mail directed to prominent United States citizens and postal facilities. Whereas the addressee was not affected, other people have died from the disease suspected to be contracted through cross-contaminated mail. The Washington Post newspaper article, dated Dec. 3, 2001, reported “A letter apparently mailed to an address near the Bronx home of anthrax victim Kathy Nguyen passed through the same New Jersey postal sorting machine within seconds of the anthrax-laced letter sent to Sen. Patrick J. Leahy (D-Vt.), officials said yesterday.” Even though none of the workers at the sorting facility had symptoms of cutaneous or inhalation anthrax or unusual absences, cross-contamination through the mail remains a serious threat to anyone that comes in contact with the mail.

There are several known methods to kill the anthrax bacterial spores. The spores can be incinerated; therefore, suspicious mail can be simply burned. The toxic chlorine dioxide gas was used to kill the mail delivered spores in U.S. Senator Daschle's office suite and liquid or foam decontaminant in the offices of 11 other senators. The gas was pumped into the office and left in place for 20 hours to be followed by another chemical to remove the gas with tests to ensure that no trace remained. The U.S. Postal Service (USPS) is sending mail to be sterilized by electron-beam machines. These machines were originally designed to sterilize medical devices and to get rid of germs in food products. The USPS is purchasing several of these machines at $5 million each for installation at the mail sorting centers to be used on person-to-person and consumer-to-business mail which account for some 40 billion pieces of mail each year. Sandia National Laboratories, operated by Lockheed Martin Co. for the U.S. Department of Energy, has created environment friendly decontamination foam that kills anthrax spores. This product is a chemical cocktail that includes an ingredient that essentially breaks the spore's armor and then another chemical destroys the material inside. Several commercial firms use the Sandia decontaminant in several products intended for home and office use. They provide a hand-washing solution to be used as a daily protective for the post office and general public.

Doctors have little experience treating anthrax on the scale of a biological attack so it is difficult to predict exactly what might happen. However, the medical community addresses the clinical recognition and management of suspected bioterrorism events through state and national level publications. Anthrax may be successfully treated with an antibiotic if anthrax is identified as the agent of disease and people that were exposed receive prompt treatment. Effective antibiotics, administered on a 60 day course, include ciprofloxacin (Cipro), doxycycline and amoxiclin.

Currently, there is little the general public can do to guard against contracting the anthrax disease where exposure through mail delivery is the greatest threat. Authorities publish that early detection, handled by public health authorities rather than individuals, is the best defense against widespread disease. The public is encouraged to stay informed through announcements by local public health officials. The Centers for Disease Control and Prevention (CDC) is a good source of information about bioterrorism. The following are the CDC's latest guidelines for handling suspicious mail:

Do not shake the suspicious package or envelope.

Do not sniff, touch or taste any contents that may have spilled out.

Do not carry the suspicious mail around and do not have others take a look at it.

Put the suspicious package or envelope on the floor or someplace where it will not fall over.

Leave the area, closing doors behind you. Tell others about the suspicious mail and keep anybody else from going into the area.

Wash your hands with plenty of soap and warm water.

If you are at work, report the incident to your supervisor, a security officer or police. If at home, call the police or sheriff's department.

Make a list of the people who were in the room when the package or letter was opened. Include all people who may have handled this mail. Give copies of the list to the police and to local public-health officials.

Further, the CDC indicates that mail is suspicious if;

Was sent by someone you do not know;

Is addressed to someone no longer at your address;

Has a hand-written address with no return address or with a return address that can not be confirmed as legitimate;

Is lopsided or lumpy;

Is sealed with excessive amounts of tape;

Is marked “PERSONAL” or “CONFIDENTIAL”; and

Has excessive postage.

Microbiologist report that micro waving the mail will not kill anthrax since microwaves work by heating water and spores have no water in them. A common clothing iron can reach sufficient temperatures to kill spores but would have to be applied for a length of time that is more likely to burn the mail than kill the spores. Also, the spores can spray out and become airborne if steam builds up inside the envelope. As a drastic measure, suspicious mail may be incinerated to kill anthrax at the obvious loss of the mail.

Unfortunately, current methods to control exposure to infectious bacteria do not provide the means for the individual mail recipient to eliminate infectious agents prior to handling the mail. Some people have responded to the threat of bioterrorism by stockpiling food, antibiotics and other goods but many people in metropolitan areas would likely have been already exposed. Some scientists say it's a giant leap from irradiating poultry or surgical instruments to decontaminating the millions of letters and packages the U.S. Postal Service delivers daily. Exposing mail to enough radiation might be slow and the energy needed to produce the radiation, possibly radioactive isotopes or devices to accelerate electrons, would be expensive. Scientist believe the procedure would not leave the mail radioactive but could cause damage to some of the contents, particularly food. There is also a danger from ozone exposure for those operating the machines. In some applications, chemicals would prove to be a more inexpensive and practical method to destroy infectious agents.

In these respects, the inventive solution departs from the conventional concepts and designs of the prior art, and in so doing provides a method primarily developed for the purpose to inexpensively and chemically eliminate infectious agents prior to mail handling by the postal facility and/or mail recipient.

### SUMMARY OF THE INVENTION

In view of the foregoing disadvantages inherent in the known procedures to handle and eradicate infectious agents, the present invention provides a method and apparatus to eliminate biological agents before the mail comes in contact with the postal facility worker and/or mail recipient.

To attain this, the present invention comprises a bio-safe device that delivers a timed application of a decontaminant in an enclosed structure, or mailbox, to destroy biological agents on contaminated mail. In the first embodiment of the invention, the bio-safe device is configured to be readily installed in the common residential mailbox. In a second embodiment of the invention, the bio-safe device is installed in the U.S. Post Office mailbox. In a third embodiment of the invention, the bio-safe device is installed in the mail sorting center.

The bio-safe device is entirely managed by the owner. The bio-safe device is chemically charged and functionally checked on a periodic basis for operability. The bio-safe device is configured to discharge the decontaminant on a daily basis for a set duration of time to effectively eliminate the biological agent after each (daily) mail delivery. Consequently, the recipient and mail carrier is made aware of the eradication process through indication of the bio-safe device activity status clearly indicated on the mailbox to facilitate safe and complete operation.

It is to be understood that the invention is not limited in its application to the details of construction and to the arrangements of the components set forth in the following description. The invention is capable of other embodiments and of being practiced and carried out in various ways. Also, it is to be understood the phraseology and terminology employed herein are for the purpose of the description and should not be regarded as limiting.

A primary object of the present invention is to provide a method and apparatus to protect the general public against exposure to infectious agents on contaminated mail.

An object is to provide the method and apparatus for the individual mail recipient to destroy infectious agents before physical contact with the mail.

Another object is to provide a method and apparatus for the postal carrier or individual mail recipient to destroy infectious agents without destroying the mail.

Another object is to provide a method and apparatus for the postal carrier or individual mail recipient to inexpensively self manage the destruction of infectious agents.

Other objects and advantages of the present invention will become obvious to the reader and it is intended that these objects and advantages be within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 shows a block diagram of the functional elements of the bio-safe device.

FIG. 2 shows the application of the first embodiment of the bio-safe device.

FIG. 3 shows a detailed configuration of the first embodiment of the bio-safe device.

FIG. 4 shows the detail of the user control elements of the first embodiment of the bio-safe device.

FIG. 5 shows an application of the second embodiment of the bio-safe device.

FIG. 6 shows an application of the third embodiment of the bio-safe device.

### DESCRIPTION OF THE PREFERRED EMBODIMENT

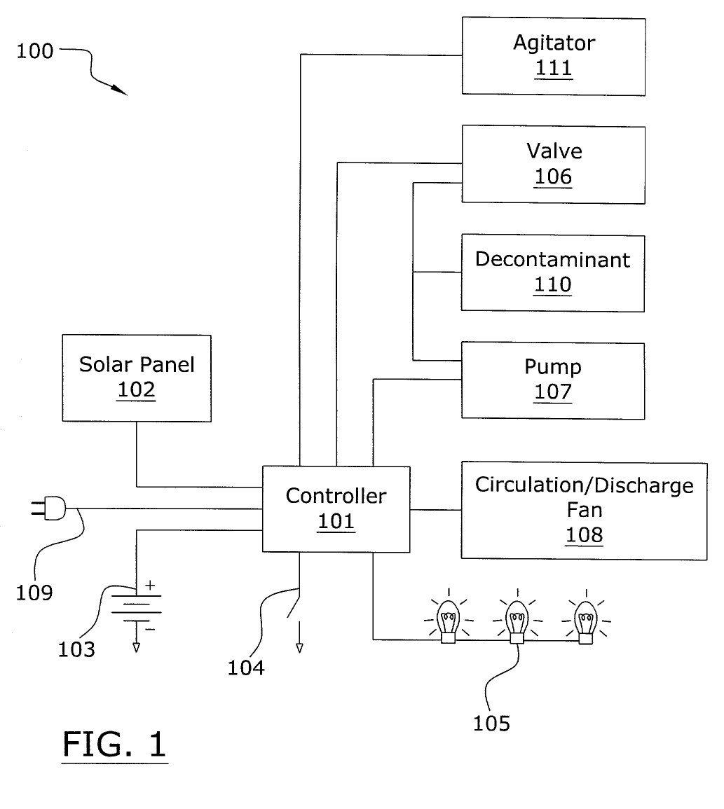

FIG. 1 shows a block diagram of the functional elements of the bio-safe device. The bio-safe device is housed in an enclosed structure, such as a common outdoor residential mailbox of the first embodiment, to effect treatment of deposited mail. A processor based controller 101 is central to the operation of the device. The controller includes a LCD front panel and pushbuttons to accept and display user inputs to define the activation periods of the device and system status. The controller depends on power delivered by a battery 103 or an optional solar panel 102 or 110 VAC line 109. For reliability, so as to not be dependable on local household power, the device preferably utilizes a rechargeable battery to source operation, recharged via the solar panel or line cord under regulatory control by the controller. The device uses a door switch 104 to signal the controller when the enclosed structure door is closed enabling normal operation. The door switch also functions to signal an audible alarm with immediate termination of an ongoing treatment cycle in the event the enclosed structure is opened. Indicator lights 105, under control of the controller 101 are also provided to indicate the bio-safe device status. A circulation fan 108 is provided to move the air/decontaminant mixture about the enclosed structure during the treatment cycle. An exhaust fan dries the mail surfaces and any residual airborne decontaminant within the enclosure through an external vent. An agitator 111 is provided to agitate the mail to ensure the entire surface of each piece of mail is effectively exposed to the decontaminant during the treatment cycle. A pump 107, to be triggered by the controller during the treatment cycle, is provided to drive a liquid based decontaminant 110 through multiple nozzles as a fine mist to cover the mail in the enclosure. FIG. 1 also depicts an electrically actuated valve 106 to alternatively control a decontaminant from a compressed cylinder of gas. The gas is released into the enclosed structure in the form of a fog to cover the mail. The valve, like the pump, is subject to timed activation by the controller.

The decontaminant is preferably Bleach in 0.55% liquid concentration since it is inexpensive, easily prepared with dilution in water and proven effective against bacterial and viral agents. Household bleach at 5% solution is diluted to 0.5% by mixing 1 part of bleach with 9 parts water. However, household bleach reduces in strength with time and would require to be replaced in the bio-safe device daily. A “stable” form of bleach solution, marketed as a hospital cleaner disinfectant, is readily available and recommended for this application to avoid daily decontaminant changes in the bio-safe device. A surface sprayed wet with this bleach concentration at room temperature will kill bacteria such as Staphylococcus aureus, Salmonella choleraesuis, Pseudomonas aeruginosa, Yersinia pestis (Bubonic Plague) and Clostridium botulinum (Botulism) in one minute. It will also kill TB (Mycobacterium tuberculosis, Hepatitis A, B and C, HIV (AIDS), Tularemia, Smallpox, fungus and other viruses in a few minutes. Labs have successfully used this concentration applied wet for several minutes at room temperature to small work areas (desks and benches) to kill anthrax.

Other commercially available decontaminants in liquid or aerosol form for use in the bio-safe device are equally effective to kill infectious agents. The decontaminant product previously discussed by Sandia was successfully used on all manner of objects and less caustic than bleach. It was found a computer keyboard worked perfectly after treatment. After about an hour, the Sandia product leaves behind a clear soapy-like film or residue that can be wiped off with towels or rinsed off with water.

In operation of the bio-safe device, the decontaminant is pulsed as a mist or aerosol fog into the enclosed structure such that the mail is completely covered for about 30 minutes to be followed by a 2 minute forced air exchange cycle to dry and discharge airborne decontaminant. Activation of the treatment cycle depends on the start and stop times programmed by the user. The cycle will delay or not start if the controller monitors that the enclosure door (door switch) is open. An activated treatment cycle is immediately terminated if the enclosure door is opened. Typically, the bio-safe device user chooses a daily activation period after the enclosed structure is loaded; the mail has been delivered and before the user removes the mail at some later convenient time. Appropriately colored indicator lights and/or the control panel readout provides a warning to a mail carrier, or anyone else intent to open the mailbox, that the treatment cycle is in process (or complete) to avoid possible exposure to any infectious agent or concentrated amounts of moving decontaminant.

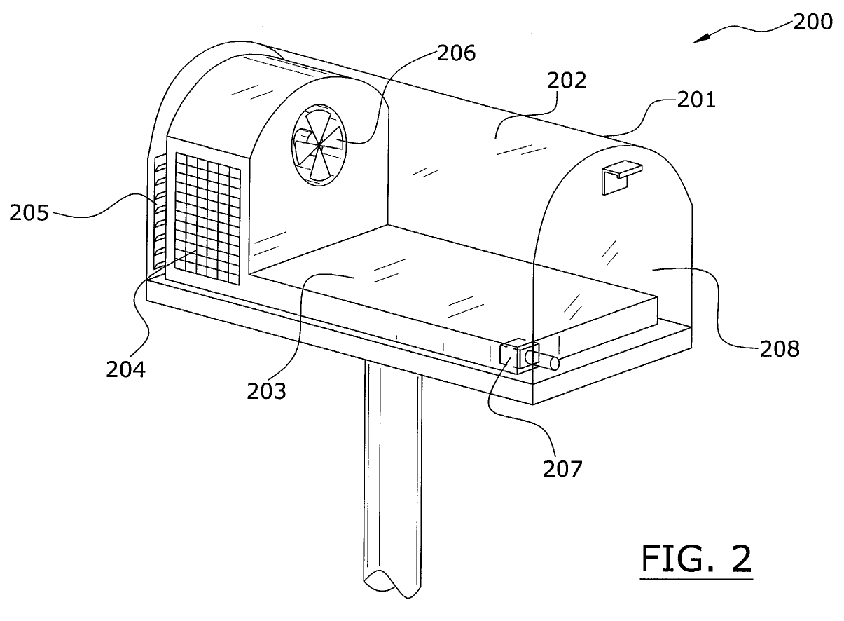

FIG. 2 shows an application of the first embodiment 200 of the bio-safe device. The bio-safe device 203 is inserted into the shape of the common household mailbox 201. Although a residential freestanding type mailbox is shown, the rectangular type generally attached to the front of a house or other designs are equally employed. To support the effectiveness of the application, the mailbox has been modified with clear (Plexiglas) sides and top 202 to allow the user to see whether or not there is mail in the box and to check the status of the bio-safe device (agitating mail or status indicator lights). The clear surface also allows sunlight to energize solar panels 204. The back of the mailbox is adapted to house a small cross section of weighted slats 205 opposite the circulation/exhaust fan 206 to facilitate the discharge of airborne decontaminant at the end of the treatment cycle. The rotation direction of the circulation fan may be reversed to also function as the exhaust fan. The exhaust fan develops sufficient air pressure to open weighted slats to ventilate the enclosure. A door switch 207 is provided to signal the bio-safe device when the mailbox door (or lid) 208 is open or closed.

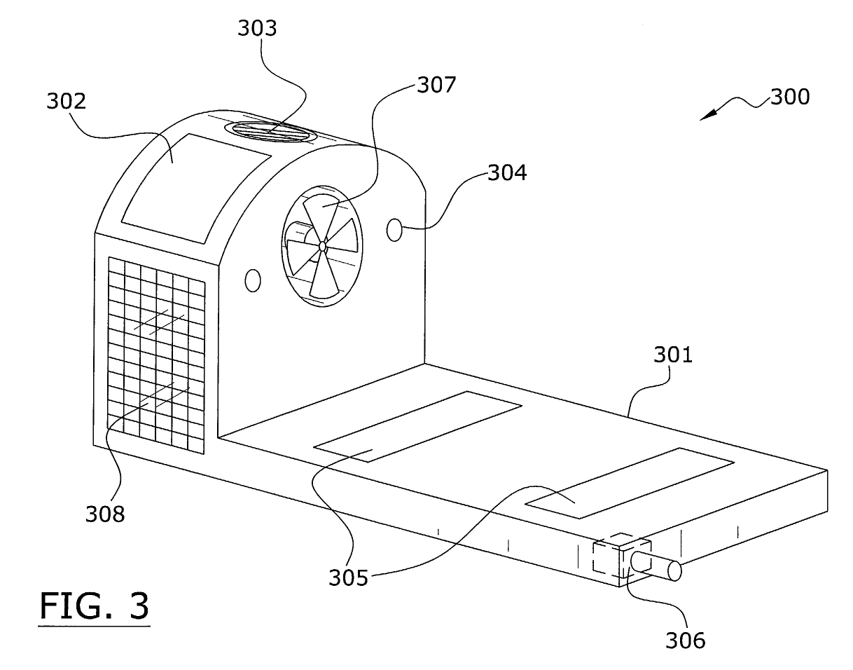

FIG. 3 shows a detailed configuration 300 of the first embodiment of the bio-safe device. The bio-safe device 301 controller control panel 302 is positioned for easy access and viewing. A cavity with liquid tight lid 303 is provided to act as a reservoir for a liquid decontaminant or aerosol canister. If the decontaminant is a liquid, the pump described for FIG. 1 would drive the nozzles 304 to create a fine mist. If an aerosol decontaminant is selected, the valve described for FIG. 1 would control the compressed decontaminant out through similar nozzles as for the liquid decontaminant. The bio-safe device also uses an agitator 305 to shift the mail to effectively expose all surfaces of the deposited mail to the decontaminant during the treatment cycle. The agitator can take many forms such as simple alternating step up bars as shown in FIG. 3 or an oscillating wire cradle. The simple mechanics to drive a reciprocating type agitator is well known in the art. A pushrod 306 is provided to mechanically transfer the open or closed position of the mailbox door (or lid) to an internal switch sensed by the controller. The pushrod may contain a longitudinal screw apparatus to adjust an overall length to correctly fit the mailbox for proper actuation of the switch. A circulation fan 307 is included to move the misted or fog decontaminant about the enclosure during the treatment cycle. The circulation fan direction may be electrically reversed to ventilate the enclosure at the end of the treatment cycle. The reversed fan, or another fan, would produce sufficient air pressure to open a vent in the mailbox as described for FIG. 2. Solar panels 308 are optionally included to augment an internal battery and positioned on the bio-safe device or enclosure to effectively capture sunlight passing through the mailbox clear wall.

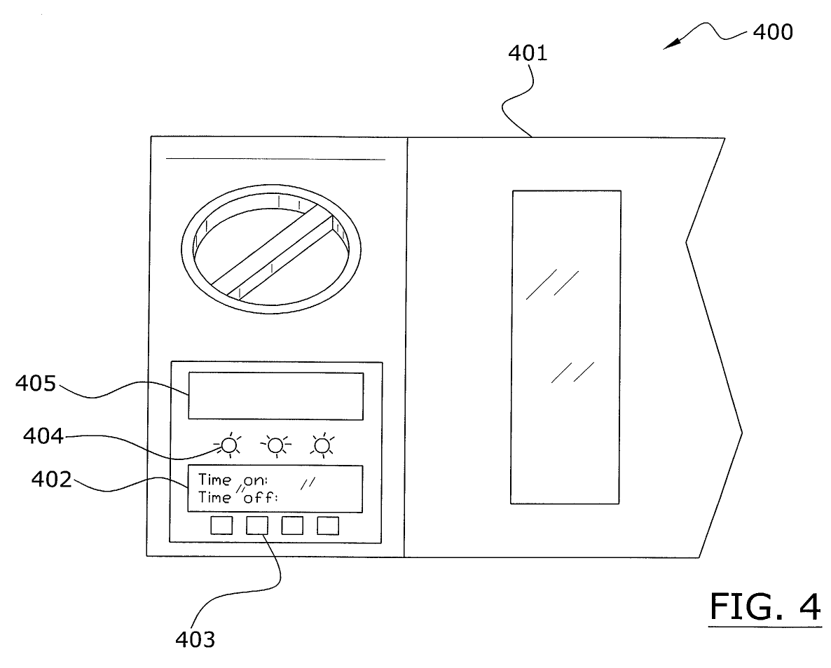

FIG. 4 shows 400 the detail of the user control elements of the first embodiment of the bio-safe device 401. A Liquid Crystal Display (LCD) 402 is provided to display user inputs through several pushbuttons 403 and display the programmed and current status of the bio-safe device. Several indicator lights 404 are also provided to also indicate various status states of the bio-safe device including a green indicator to indicate the system is ready and functional, a yellow indicator to indicate the device is engaged in a treatment cycle and a red indicator to indicate a low level of decontaminant. A water tight battery compartment 405 is placed for easy access by the user. Battery charge status may be indicated by an indicator light or on the LCD. The LCD screen and pushbuttons may alternatively be replaced with a pair of accessible variable resistant pots to set the daily start time and period for the treatment cycle where the indicator lights would provide sufficient bio-safe device operability status.

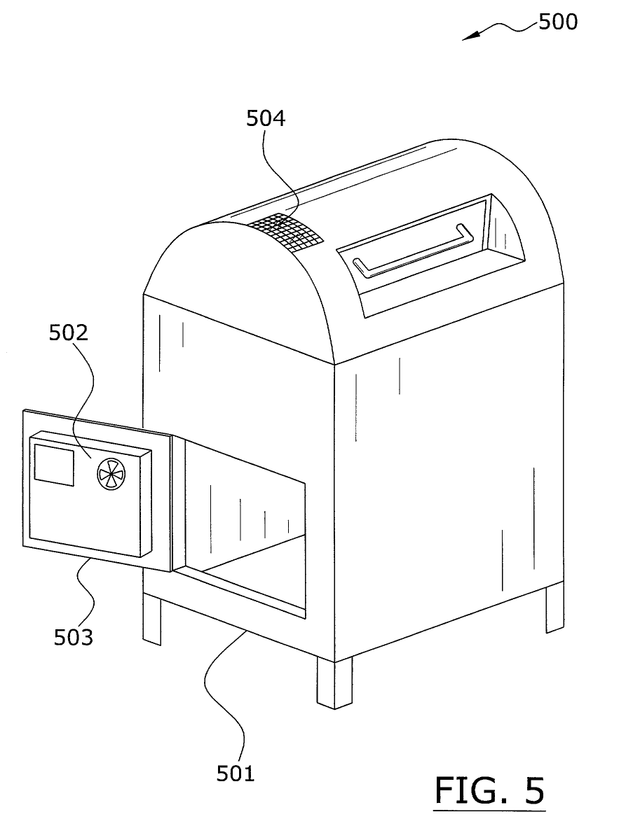

FIG. 5 shows an application 500 of the second embodiment of the bio-safe device. The bio-safe device is functionally the same as discussed for FIGS. 1-4 but physically adapted for the larger U.S. Post Office mailbox 501. The bio-safe device can also be adapted to other postal service drop boxes. The bio-safe device 502 is mounted to the inside of a door 503 for protection when closed and for easy access when open. An optional solar panel is mounted on a southern exposed surface of the mailbox 504, perhaps the door, to augment the battery. In this application, the mailbox manager would program the bio-safe device to perform a treatment cycle prior to the scheduled mail pick-up. Postal boxes generally include a mail door that blocks access to the interior of the box when open; consequently, this configuration will protect the postal box user if accessed during a treatment cycle.

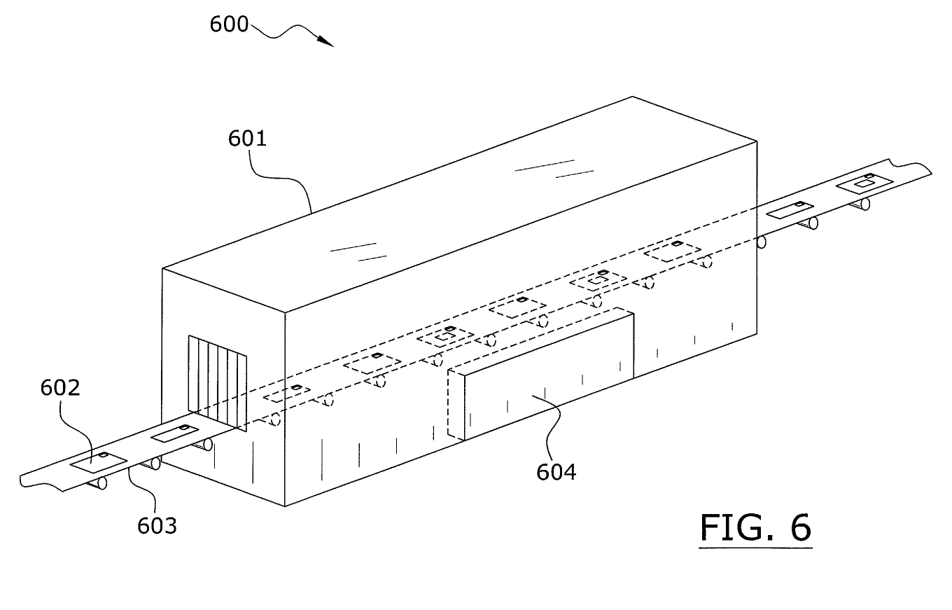

FIG. 6 shows an application 600 of the third embodiment of the bio-safe device housed in a postal mail sorting facility receiving box 601. This application is intended for use at the mail entry point of a mail handling facility. The bio-safe device discussed in FIGS. 1-4 is physically reconfigured 604 to decontaminate mail 602 in a large enclosure or small room. Mail is moved through the treatment enclosure on a speed controlled conveyor belt 603 with physical shifting of the mail to ensure sufficient coverage and coverage time by the decontaminant. The processing unit includes design advantages to facilitate the treatment cycle including barrier strip doors to minimize the egress of decontaminant, clear panels for process inspection and filtered exhaust vent to manage residual decontaminant. The bio-safe device and conveyor belt are necessarily programmed to serially treat large amounts of mail.

It will be appreciated that in general, the inventive product decontaminates cross contaminated mail. The inventive solution destroys biological agents prior to the mail carrier or individual mail recipient handling the mail thus eliminating exposure and a dependence on belated public warning notices or early disease detection.

While specific embodiments of the invention have been described and illustrated, such embodiments should be considered illustrative of the invention only and not as limiting the invention as construed in accordance with the accompanying claims. As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed to be within the expertise of those skilled in the art, and all equivalent structural variations and relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A method to eliminate biological agents from contaminated mail comprising;
depositing mail in an enclosure;
covering the mail with a chemical decontaminant;
destroying biological agents; and
removing mail from the enclosure.

2. A method as claimed in claim 1 wherein said enclosure comprises a resident mailbox, postal service mailbox and postal mail sorting facility receiving box.

3. A method as claimed in claim 1 wherein said covering the mail with a chemical decontaminant further comprises shifting the mail to expose additional mail surface area to said decontaminant.

4. A method as claimed in claim 1 wherein said covering the mail with a chemical decontaminant further comprises the mail is covered by a misted form of the decontaminant or a fog form of the decontaminant.

5. A method as claimed in claim 1 wherein said chemical decontaminant comprises a mixture of bleach and water.

6. A method as claimed in claim 5 wherein said mixture of bleach and water comprises about 0.5% bleach.

7. A method as claimed in claim 5 wherein said decontaminant comprises a mixture of stable bleach and water.

8. A method as claimed in claim 1 wherein said biological agents comprise Bacillus anthraces (anthrax).

9. A method as claimed in claim 8 wherein said biological agents further comprises Staphylococcus aureus, Salmonella choleraesuis, Pseudomonas aeruginosa, Yersinia pestis (Bubonic Plague), Clostridium botulinum (Botulism), Mycobacterium tuberculosis (TB), Hepatitis A, B and C, HIV (AIDS), Tularemia and Smallpox.

10. A method as claimed in claim 1 further comprising drying residual decontaminant and removing airborne decontaminant in said enclosure after said destroying biological agents.

11. A method as claimed in claim 1 wherein said depositing mail in an enclosure further comprises cross-contaminated mail.

12. An apparatus to eliminate biological agents from contaminated mail comprising;
an enclosure to contain deposited mail; and
a bio-safe device to perform a treatment cycle of the mail with a chemical decontaminant to destroy biological agents.

13. An apparatus as claimed in claim 12 wherein said enclosure comprises a resident mailbox, postal service mailbox and postal mail sorting facility receiving box.

14. An apparatus as claimed in claim 12 wherein said bio-safe device further comprises;
a controller to control the treatment cycle;
a control panel to receive user control inputs and display system status;
a pump to drive the chemical decontaminant into said enclosure;
a first fan to circulate the decontaminant through said enclosure;
a second fan to dry decontaminant and exhaust airborne decontaminant;
indicator lights to indicate the status of said bio-safe device;
a battery to power said bio-safe device; and
a door switch to signal the controller whether the enclosure door is open or closed.

15. An apparatus as claimed in claim 14 further comprises a valve to control the flow of compressed gas or aerosol decontaminant.

16. An apparatus as claimed in claim 14 further comprises an agitator to shift the mail to expose additional surface area of the mail to the decontaminant during the treatment cycle.

17. An apparatus as claimed in claim 14 where the first fan and second fan are the same fan.

18. An apparatus as claimed in claim 14 wherein said biological agents comprise Bacillus anthraces (anthrax).

19. An apparatus as claimed in claim 14 wherein said biological agents further comprises Staphylococcus aureus, Salmonella choleraesuis, Pseudomonas aeruginosa, Yersinia pestis (Bubonic Plague), Clostridium botulinum (botulism), Mycobacterium tuberculosis (TB), Hepatitis A, B and C, HIV (AIDS), tularemia and smallpox.

20. An apparatus as claimed in claim 14 where said indicator lights indications comprise the system is functional, the bio-safe device is engaged in a treatment cycle and the decontaminant level is low.

21. An apparatus as claimed in claim 14 where said battery is a rechargeable battery.

22. An apparatus as claimed in claim 21 further comprises a solar panel to provide power to operate said bio-safe device and charge said battery.
